(19) United States
(12) Patent Application Publication
(10) Pub. No.: US 2017/0196219 A1
(43) Pub. Date: Jul. 13, 2017

(54) Use of Allene Oxide Synthase for Semen Preservation and Assisted Reproduction

(71) Applicant: PACIFIC BRANDS, Auckland (NZ)

(72) Inventors: Ralph Andrew Backhaus, Phoenix, AZ (US); Steven Charles Hodgkinson, Hamilton (NZ)

(21) Appl. No.: 15/314,428

(22) PCT Filed: May 27, 2015
(86) PCT No.: PCT/NZ2015/050067

§ 371 Date: Nov. 28, 2016

Related U.S. Application Data
(60) Provisional application No. 62/004,527, filed on May 29, 2014.

(51) Int. Cl. A01N 1/02; A61D 19/02; A61K 38/51; C12N 9/88; A61K 35/52; A61K 36/28

(52) U.S. Cl. CPC A01N 1/0226; A01N 1/0284; A61K 35/52; A61K 36/28; A61K 38/51; C12Y 402/01092; C12N 9/88; A61D 19/02

(57) ABSTRACT

The use of allene oxide synthase as a preservative for semen particularly for use in assisted reproductive procedures for humans and in the breeding of cattle horses and other animals.

### TECHNICAL FIELD

The Invention relates to the use of the enzyme allene oxide synthase in the general field of assisted reproductive technology. In particular, the Invention relates to improving the stability of semen (and sperm) for use in assisted fertlllsatlon procedures including artificial insemination, in vitro fertilisatlon, sperm sexing and in the in vitro manipulation of oocytes in order to improve their viability and fertility. The Invention relates to gametes from humans, cattle, horses, pigs, poultry and other animals.

### BACKGROUND OF THE INVENTION

Allene oxide synthase (AOS) is a cytochrome p450 enzyme family member (CYP74A; EC 4.2.1.92) first isolated from the guayule rubber plant Parthenlum argentatum (GenBank CAA55025.2). Also known as the guayule rubber particle protein (RPP), AOS has been purified and cloned from the guayule rubber plant (U.S. Pat. No. 5,633,433 and U.S. Pat. No. 6,132,711).

AOS is an antioxidant enzyme with specificity for lipid peroxides in biological systems. As reported in U.S. Pat. No. 6,132,711, AOS rapidly converts free or esterified fatty acid peroxides or hydroperoxides into their corresponding epoxides which are, in turn, converted to ketols. The lipid peroxide and hydroperoxide substrates for this enzyme are said to be toxic to biological organisms and can generate additional peroxides by chain propagation reactions as well as causing oxidative damage to proteins and DNA. In the presence of AOS these compounds are rapidly converted to epoxides and the chain reaction is broken.

The ketol species produced by the action of AOS are relatively biologically inert compared to lipid peroxides and so U.S. Pat. No. 6,132,711 speculates that the antioxidant effect of AOS may be useful in a variety of applications. These applications include the preservation of plant seeds, the treatment of trauma patients with severe blood loss, the promotion of apoptosis in the treatment of certain diseases, inducing resistance to herbicides in tobacco plants, increasing the life span of sperm used for artificial insemination or In vitro fertilisation, and for increasing the average and maximum life span of biological organisms. However, these uses are entirely speculative having no experimental support in U.S. Pat. No. 6,132,711.

Many agents, whether single compound or multiple ingredient substances, whether derived from natural biological sources or manufactured synthetically, are known to exhibit antioxidant behaviour. Some are broad spectrum antioxidants in that they exhibit an antioxidative effect in a range of biological systems, whereas others are selective and have an antioxidant effect in a specific set of biological parameters.

Additionally, antioxidants may be categorised as being of the ‘suicide’ variety (meaning one molecule of antioxidant is consumed in neutralising one molecule of a reactive oxidative species) or may be categorised as enzymic in which a single molecule of enzyme may neutralise many oxidative molecules.

AOS has been shown to be of benefit in the treatment of severe ischemic injury and ischemia-reperfusion injury (U.S. Pat. No. 7,157,082). Ischemic injury is caused by a traumatic event, such as stroke or heart attack, which leads to a lack of blood supply causing a shortage of oxygen to tissue. Ischemia-reperfusion refers to the tissue damage caused when blood supply returns to the tissue after a period of ischemia or lack of oxygen.

The stability of semen (as well as sperm and ova) is widely recognised as a very significant constraint in the animal artificial insemination and assisted reproductive technologies sector. The Instability of semen adds to cost, inefficiencies and logistical complexities. This is the case for both fresh semen and frozen semen, and across a range of species besides the bovine cattle breeding sector. Frozen sperm suffers from the problem of loss of viability over the freezing and thawing steps, and fresh semen is severely constrained by a very limited shelf life (˜3 days). Industry has managed the stability issue through a range of measures for improving efficiencies. For example, incremental improvements in diluent composition and processing are aimed at improving survival. Nonetheless, it is widely recognised that semen stability remains a major issue and there is an ongoing need to find ways to improve semen performance. Additionally, the more advanced semen processing technologies, such as those involving sorting of sperm on the basis of whether they are X or Y (female or male), provide additional challenges to sperm viability as a result of the methodologies employed. These are often oxidative in nature occurring as a result of thermal, pressure, light, chemical or biochemical insults to the cells during processing.

The applicant has now found that AOS is an effective stability enhancer for both fresh and frozen semen. Accordingly, it is an object of the Invention to provide a new way to extend the functional life of semen for use in assisted reproductive procedures for humans and other animals, or at least to provide a useful alternative to existing methods.

### SUMMARY OF THE INVENTION

In one aspect the Invention provides the use of allene oxide synthase as a preservative for semen. The allene oxide synthase may be, or may have been cloned from, allene oxide synthase from the guayule rubber plant Parthenlum argentatum. The allene oxide synthase preferably has the amino add sequence of SEQ ID No. 1 or is a functionally equivalent variant thereof.

The allene oxide synthase may be in any suitable form for use, for example in a pH-buffered aqueous medium. The allene oxide synthase may be present in the medium at any suitable concentration, for example 1 to 10 μg/ml.

In some embodiments of the Invention, the semen is bovine semen. In some other embodiments of the Invention, the semen is human semen. In other embodiments, the semen may be from a horse, sheep, pig or poultry.

In a second aspect the Invention provides a method of extending the life of semen by contacting the semen with allene oxide synthase. Typically, the semen is stored in the presence of allene oxide synthase.

In certain embodiments of the Invention, the allene oxide synthase is added to the semen and the semen is frozen, stored for a period of time, and then thawed before use. In other embodiments, the semen is fresh semen.

Preferably the life of the semen is extended such that at least 50% of sperm in the semen are viable 4 days from ejaculation.

In a further aspect the Invention provides semen containing allene oxide synthase. The semen may be frozen or fresh.

In another aspect the invention provides the use of the semen containing allene oxide synthase in an artificial insemination process, an in vitro fertilisation process, or a sperm sorting process. The artificial insemination process includes the step of artificially inseminating a human female, a bovine cow, a horse, a pig, a sheep, or poultry.

In yet another aspect the Invention provides a composition for preserving semen which comprises an effective amount of allene oxide synthase.

### BRIEF DESCRIPTION OF THE FIGURES

FIG. 1 shows the mean relative survival of sperm in diluented bull semen stored using standard ‘fresh’ methodologies for four days.

FIG. 2 shows the effect of AOS on sperm motility of frozen bull semen.

FIG. 3 shows the effect of AOS on sperm motility performance.

FIG. 4 is the amino add sequence SEQ ID No. 1.

### DETAILED DESCRIPTION

The Invention relates generally to the use of AOS as a preservative for semen. The Invention also relates to semen containing AOS as a preservative, to the use of the semen containing AOS in an artificial insemination process, an in vitro fertilisation process, or a sperm sorting process, and a composition for preserving semen which comprises AOS.

It will be appreciated that any reference in this specification to the preservation of semen means extending its functional life beyond the functional life of untreated or unprocessed semen, and Includes the preservation of the sperm in that semen.

The term “allene oxide synthase” or “AOS” as used in this specification is intended to mean any enzyme that converts lipoxygenase-derived fatty add hydroperoxides to allene epoxides (which are precursors of the growth regulator jasmonic add in plants), and Includes for example the allene oxide synthase isolated from the rubber plant Parthenlum argentatum. The term also includes any functionally equivalent peptide or protein of an AOS, and Includes AOS obtained from any source or by any method, for example by chemical synthesis and/or gene expression or cloning techniques. Any reference to AOS in this specification should be taken to include reference to functionally equivalent variants thereof, unless otherwise indicated.

The term “functionally equivalent variant” as used in this specification Includes those peptides or proteins having one or more (for example 1 to 50, 1 to 30, 1 to 20, 1 to 10 or 1 to 5) deletions, additions and/or substitutions while substantially retaining the desired function of the AOS or to variants that are derivatised by chemical modification of selected amino adds or the overall amino add structure. Amino add substitutions will typically be conservative amino add substitutions. It should be appreciated that a functionally equivalent variant may have a level of activity higher or lower than the protein of which it is a variant. In various embodiments of the invention, a functionally equivalent variant has at least 50%, at least 60%, at least 70%, at least 80%, at least 90%, at least 95%, or at least 99% of the level of activity of the protein of which it is a variant. Functionally equivalent variants will have antioxidant activity. For example, they may have the ability to convert lipid peroxides/hydroperoxides to lipid epoxides at the lipid/cell membrane interface.

In some embodiments of the Invention, the AOS is the AOS described in Genbank CAA55025.2 or a functionally equivalent variant of this protein.

Those skilled in the field will readily be able to assess the function and determine the level of activity of a protein based on the Information in this specification and using known techniques. By way of example, antioxidant activity can be determined using the methods described in Pinchuk et al., Chemistry and Physics of Lipids, 164 (2001), 42-48, or using a commercially available assay kit available thought Sigma-Aldrich.

The term “conservative amino add substitutions” as used in this specification is intended to mean the substitution of amino acids that have similar biochemical properties. It will be appreciated that appropriate conservative amino add substitutions are based on the relative similarity between different amino acids, including the similarity of the amino add side chain substituents (for example their size, charge, hydrophilcity, hydrophobicity and the like). By way of example, a conservative substitution includes substitution of one aliphatic amino add for another aliphatic amino acid, substitution of an amino add having an hydroxyl- or sulphur-containing side chain with another amino add having an hydroxyl- or sulphur-containing side chain, substitution of an aromatic amino add with another aromatic amino add, substitution of a basic amino add with another basic amino add, or substitution of an acidic amino add with another acid amino add. Examples of conservative amino add substitutions include:
substitution of glycine, alanine, valine, leucine, or isoleucine, one for another
substitution of serine, cysteine, theronine, or methionine, one for another
substitution of phenylalanine, tyrosine, or tryptophan, one or another
substitution of histidine, lysine, or arginine, one for another
substitution of aspartic acid, glutamic add, asparagine or glutamine, one for another

The AOS of the Invention may be isolated from natural sources, or derived by chemical synthesis (for example, fmoc solid phase peptide synthesis as described in Fields G. B., Lauer-Flelds J. L., Liu R. Q. and Barany G., (2002) Principles and Practice of Solid-Phase peptide Synthesis; Grant G., (2002) Evaluation of the Synthetic Product. Synthetic Peptides, A User's Guide, Grant G. A., Second Edition, 93-219; 220-291, Oxford University Press, New York) or genetic expression techniques. Standard recombinant DNA and molecular cloning techniques are described for example in Sambrook, and Maniatis, Molecular Cloning: A Laboratory Manual, Second Edition, Cold Spring Harbor Laboratory Press, Cold Spring Harbor, N.Y. (1989); Silhavy et al., Experiments with Gene Fusions, Cold Spring Harbor Laboratory Press, Cold Spring Harbor, N.Y. (1984); and Ausubel et al., Current Protocols in Molecular Biology, published by Greene Publishing Assoc. and Wiley-Interscience (1987). The production of a protein or peptide for use in the Invention by an appropriate transgenic animal, microbe, or plant is also contemplated.

The AOS may be connected to one or more additional compounds. For example, it may be connected to a compound that aids the function or activity of the AOS, protects the AOS from degradation, otherwise improves its half-life, aids in isolation and/or purification of the AOS during manufacture (for example ubiquitin, a his-tag, or biotin), or assists with cell membrane translocation or cell-specific targeting. These additional compounds may include, for example, peptides, nucleic adds, lipids and carbohydrates.

The additional compounds may be connected to the AOS, or synthesised as a part of a construct, using any appropriate means which allows the AOS to retain at least a level of Its desired function. The term “connected” should be taken broadly to encompass any form of attachment, bonding, fusion or association between the AOS and the compound (for example, covalent bonding, ionic bonding, hydrogen bonding, aromatic stacking interactions, amide bonds, disulfide bonding, chelation) and should not be taken to imply a particular strength of connection. The AOS and the compound may be connected in an Irreversible or a reversible manner, such that upon administration the AOS is released from the compound.

Since AOS is an enzymatic antioxidant, it provides an additional benefit over traditional chemical/non-enzymatic antioxidants, such as vitamin E, in that AOS is capable of catalysing thousands of antioxidation reactions per AOS molecule. In contrast, most chemical/non-enzymatic antioxidants only have the ability to take part in one reaction. Consequently, AOS is effective at very low concentrations. Other antioxidants that must be used at much higher concentrations (typically orders of magnitude higher) may have toxicity problems.

Further, virtually all antioxidant enzymes, such as catalase and superoxide dismutase, also generate a secondary pro-oxidant radical species that requires a second enzyme to remove it. AOS is believed to act against lipid hydroperoxides and is able to act alone, i.e. In the absence of a second enzyme.

The applicant has found that AOS is an effective preservative of semen, both fresh semen and frozen semen. AOS therefore has potential application in assisted reproduction procedures including for humans and in animal breeding industries, particularly cattle and horse breeding.

In respect of the cattle breeding and dairy industries in New Zealand alone, it is estimated that of 100 cows artificially inseminated 70 will not return and approximately 60 will be in calf. While various factors including infertility of the cows themselves may account for the remaining 30 It is also widely recognised that semen quality is a key consideration perhaps accounting for 10-15 of non-pregnant animals. In financial terms, a 1% gain in fertility is estimated to be worth $4M to the New Zealand dairy industry alone. Progress with the Infertility problem is therefore especially relevant to artificial insemination and related semen processing industries such as those involved in X/Y sorting. In addition to improving the pregnancy success rate for cows, improvements in semen quality can also be expected to result in cost benefits in other ways, such as a reduction in the number of required dosages for fertilisation with concomitant increases in straws per ejaculate.

With respect to assisted reproductive technologies for humans, AOS may be useful as an additive to ova culture media during in vitro fertilisatlon (IVF) procedures and for the stabilisation of sperm prior to intracytoplasmic sperm injection (ICSI) extending to the embryo culture, sorting, storage and transfer process.

While it is likely that there are several mechanisms of sperm cell death in an animal ejaculate, notably the two main ones, apoptosis and oxidative stress, each have lipid peroxidation as a central step in the causal pathway. In these mechanisms oxidative damage to lipid constituents of cell membranes including mitochondrial membranes results in the loss of membrane integrity, cellular dysfunction and necrosis. Importantly, the biochemistry of these processes involves oxidation of membrane lipids to lipid peroxides which then cascade on to cause oxidative damage of cellular proteins, DNA and other lipids. This oxidative pathway to cell death is also important in situations where cells are exposed to significant environmental challenges including heating/cooling, freezing/thawing, shear forces and light exposure.

The role of oxidative damage has been recognised by industry for some time leading to the development of antioxidative additives for semen and IVF diluents with some success. One example is Caprogen™ which is a protein extract of bovine liver containing a catalase enzyme. The use of Caprogen™ is costly and, as a bovine offal extract, faces restrictions in export markets.

The AOS will ordinarily be used in the form of an aqueous composition that includes a buffer, and a semen extender. The extender typically consists of a diluent buffer, protein and lipid components. The AOS may be present at any suitable concentration, for example in the range 1 to 10 μg/ml. Alternative ranges include, but are not limited to, any range of integers within 1 to 10 including any range having at it's lower limit 1, 2, 3, 4, 5, 6, 7, 8, 9 or 10 μg/ml and at it's upper limit 1, 2, 3, 4, 5, 6, 7, 8, 9 or 10 μg/ml.

The applicant has determined that AOS was able to increase the survival of sperm in fresh bull semen by ˜50% after 4 days.

Example 1 shows that AOS is an effective preservative of fresh bull semen. At a concentration of 3 μg/ml, AOS increased the survival of sperm by approximately 50% after 4 days. An improvement of this order of magnitude is significant for several assisted reproductive technology processes. It will be appreciated that the short ‘shelf life’ of fresh untreated semen places significant constraints on its use especially if, for example, this first requires transport before use. For this reason an increase in sperm survival of 50% 4 days after collection is economically important for cattle breeding because it markedly increases the window of opportunity for use of the semen to achieve its intended use of fertilising cows. Similar improvements to the functional life of semen from other animals, including humans, can be expected.

Example 2 confirms that AOS has a marked positive effect on maintaining sperm motility post-thaw. The parameters examined include survival and motility post-thaw, and enzyme dose/response effects. The applicant determined that the AOS significantly increased post-thaw survival (P<0.05) and lifted motility by ˜120% 24 h post-thaw (P<0.01) at an enzyme concentration of 2 μg/ml. Additionally, more sperm were found to be membrane intact and available for fertilisatlon. Overall from the studies, it can be concluded that AOS is effective at improving the tolerance of bovine sperm to dilution, freezing and post-thaw processes. In the case of cattle breeding, this means that many more straws per ejaculate can be used thereby increasing the value of each ejaculate to the breeding industry.

Ovulation is a very discrete biological event in females, including dairy cows, and for fertilisation to occur the ova must encounter viable semen in the short period of Its functional life. It will be appreciated that this is more likely to occur if the sperm retains its motility for longer after insemination so as to increase the probability that it is able to propel itself to the point of contact with the ova. Extended motility at 24 h is therefore a strong indication that the overall viability (fertility) of AOS treated semen can be expected to be greater. In cattle breeding, the number of animals that fall to become fertilised will therefore be lower and this, in turn, can be expected to decrease costs and Increase the value of AOS treated semen.

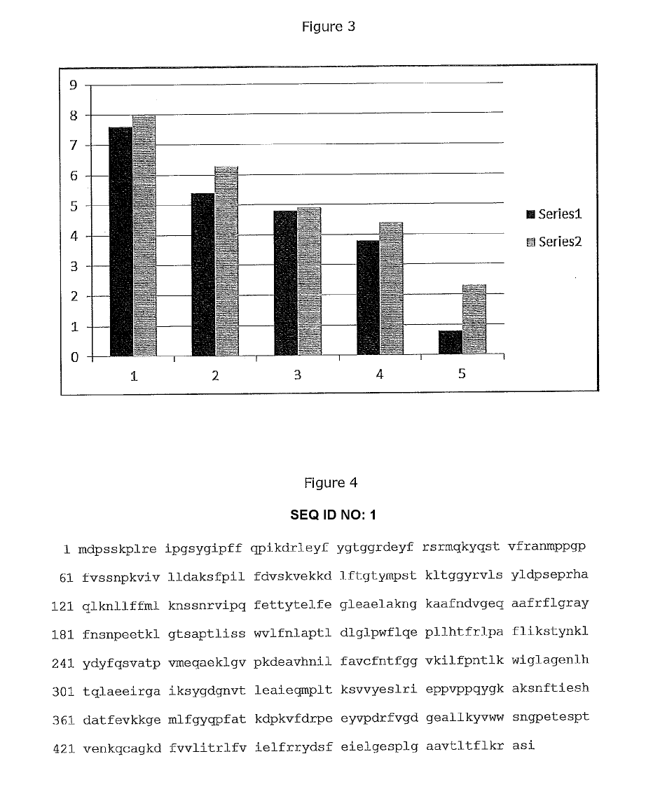

Example 2 shows a decline in sperm motility (FIG. 2) and in an index of sperm motility performance (migratory efficiency, FIG. 3) over time post-thaw. In particular, the results for AOS at 2 μg/ml show motility trending to significance across the time course. By 24 hours post-thaw, motility is more than double the control levels (+120%) and the difference is highly significant (P<0.01). Additionally, the effect of AOS was shown to be dose responsive and maximal at an enzyme concentration of 2 μg/ml. The positive effect of AOS was even more pronounced in the Motility Index analyses (FIG. 3), lifting this measure of sperm performance by 2.9 fold after 24 hours.

AOS dearly has a significant positive effect on both sperm survival and measures of sperm motility which are known key indicators of sperm performance and fertility. Additionally, more sperm were found to be membrane intact and available for fertilisation following AOS treatment. At high dilutions (2 μg/ml), AOS was found to be effective at improving the tolerance of bovine sperm to dilution, freezing and post-thaw processing in a frozen semen process and the performance of semen in a ‘fresh’ process.

Example 3 shows that there was no effect on the total number of oocytes developing to embryos, but there was a significant effect on the number of transferable quality embryos produced from both the number of oocytes becoming mature and the number that cleaved. The data indicate that AOS has no deleterious effects on oocyte developing to embryos and Increases the ‘yield’ of embryos that develop to maturity in IVF procedures.

As used in this specification, the words “comprises”, “comprising”, and similar words, are not to be interpreted in an exclusive or exhaustive sense. In other words, they are intended to mean “Including, but not limited to”.

The Invention is further described with reference to the following examples. It will be appreciated that the Invention as claimed is not intended to be limited in any way by these examples.

Any reference to prior art documents in this specification is not to be considered an admission that such prior art is widely known or forms part of the common general knowledge in the field.

### EXAMPLES

### Example 1—Fresh Bull Semen

This study was performed using ejaculates from 5 bulls using well established procedures. Semen was collected using an artificial vagina at 42° C. and transferred to the lab for initial quality assurance (volume, motility, sperm concentration). Samples were then diluted to a standard sperm concentration of 10×10⁶ spermatozoa per ml at 37° C. using each of the experimental extenders. The experimental extenders comprised sodium citrate 1.856 g per 100 ml, glucose 1 g per 100 ml, egg yolk 20 ml per 100 ml containing antibiotic preservatives, and were adjusted to pH 7.0. Experimental additives were then added to this extender base as shown in 1-5 below. Diluted samples (20 ml) were then cooled to 5° C. at 0.25° C. per minute and stored at 5° C. for 5 days. Samples were withdrawn at Day 1 and Day 4 and percentage sperm survival and motility index determined using standard procedures.

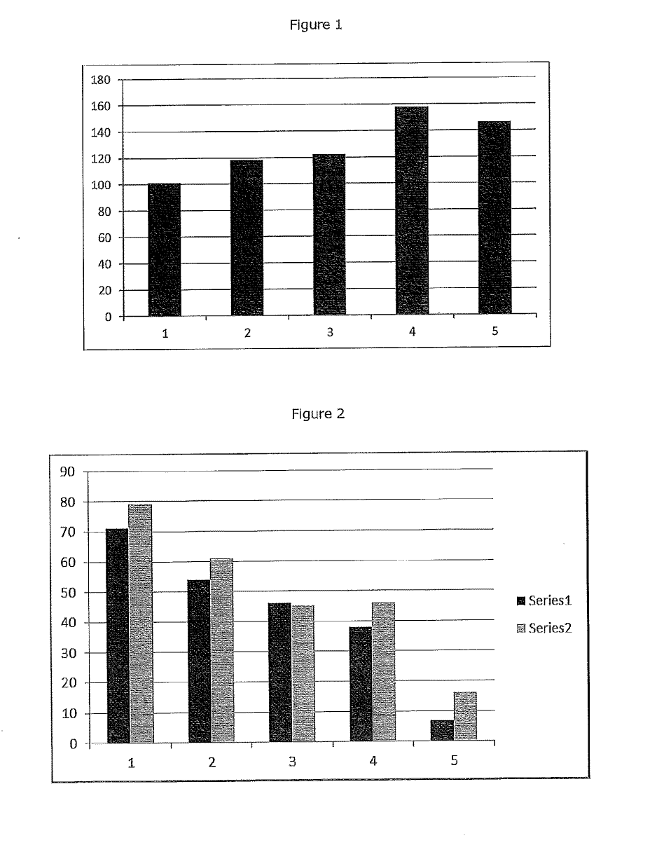

The results are shown in FIG. 1, where:

1. Control—Diluent only

2. Catalase—Diluent plus 10 μg/ml catalase

3. Caprogen™—Diluent plus Caprogen™ at standard concentration

4. AOS—Diluent plus 3 μg/ml AOS

5. AOS—Diluent plus 1 μg/ml AOS

FIG. 1 shows the mean relative survival of sperm in diluented bull semen stored using standard ‘fresh’ methodologies for four days with and without test additives. Overall control levels of survival at 4 days were ˜55% of day 0. The results are normalised against the control. Thus, an AOS concentration of 3 μg/ml AOS results in a ˜50% improvement in survival compared to the control. This difference is highly significant P<0.01. At an AOS concentration of 1 μg/ml, the difference reached significance at the 0.05 level. The results obtained with catalase and Caprogen™ were not significant.

### Example 2—Frozen Bull Semen

This study was conducted to determine whether AOS can improve the tolerance of bovine sperm to the freeze-thaw procedure. Post-thaw motility was measured. Semen samples from 5 bulls were diluted into media to standard concentration and each divided into 2 aliquots. AOS was added to one aliquot to a final concentration of 2 μg/ml, with the second aliquot from each bull acting as a control. The semen was the dispensed into straws for freezing. The temperature of the straws was then reduced using standard procedures and the straws stored in liquid nitrogen. After thawing at room temperature, the straws were allowed to oxygenate using standard procedures. The straws were then stored at room temperature for up to 24 h prior to evaluation for survival, motility and motility index.

Sperm motility over time post-thaw is shown in Table 1 and FIG. 2. % Motility refers to the percentage of the total sperm showing movement. The control treatment is the diluent and extender without AOS (Series 1). AOS treatment refers to the diluent and extender with 2 μg/ml AOS (Series 2). In FIG. 2, 1 is % motility pre-freezing, 2 is 0 hr post-thaw motility, 3 is 3 hr post-thaw motility, 4 is 6 hr post-thaw motility, and 5 is 24 hr post-thaw motility.

TABLE 1
Sperm motility

| | | Initial | 0 hr | 3 hr | 6 hr | 24 hr |
|---|---|---|---|---|---|---|
| | Treatment | Motility | Mot | Mot | Mot | Mot |
| Average | Control | 71 | 54 | 46 | 38 | 7 |
| | AOS | 79 | 61 | 45 | 46 | 16 |
| | 2 μg/ml | | | | | |
| SEM | Control | 4.6 | 6.2 | 8.6 | 10.4 | 3.4 |
| | AOS | 3.3 | 5.8 | 9.1 | 10.3 | 4.8 |
| | 2 μg/ml | | | | | |

Motility Index (relative proportion of motile sperm showing forward propulsion) is shown in Table 2 and FIG. 3. In FIG. 3, 1 is Motility Index (MI) pre-freezing, 2 is 0 hr post-thaw MI, 3 is 3 hr post-thaw MI, 4 is 6 hr post-thaw MI, and 5 is 24 hr post-thaw MI.

TABLE 2
Sperm motility index

| | | Initial | | | | |
|---|---|---|---|---|---|---|
| | | Motility | 0 hr | 3 hr | 6 hr | 24 hr |
| | Treatment | Index | MI | MI | MI | MI |
| Average | Control | 7.6 | 5.4 | 4.8 | 3.8 | 0.8 |
| | AOS | 8 | 6.3 | 5.2 | 4.4 | 2.3 |
| | 2 μg/ml | | | | | |
| SEM | Control | 0.4 | 0.6 | 0.6 | 0.9 | 0.4 |
| | AOS | 0.3 | 0.7 | 0.7 | 0.7 | 0.4 |
| | 2 μg/ml | | | | | |

### Example 3—In Vitro Embryo Culture

This experiment examined the effect of three concentrations (0.1 μg/ml, 1 μg/ml and 10 μg/ml) of AOS on embryo development and quality in an in vitro embryo production system. Standard in vitro embryo production procedures were used for maturation, fertilisation and culture of embryos. Small drops (20-50 μL) of a medium comprising embryo culture buffer or diluent in a petri dish were overlaid with mineral oil. The Initial concentration of AOS in the medium was adjusted to give the correct final concentration after the addition of oocytes, zygotes or embryos to the drops. Following culture, the embryos were assessed morphologically at day 7, and the stage and grade of embryos were recorded as per the IETS (International Embryo Transfer Society) manual for grading embryos. The proportional data for development were analysed using Binomial Generalised linear model. The embryo development data is summarised in Table 3.

TABLE 3
Development of transferable grade IVF embryos

| | | | Grade 1 & 2 | | Grade 1 & 2 | |
|---|---|---|---|---|---|---|
| | | | of total | | of cleaved | |
| Treatment¹ | N | No. cleaved | % | SE | % | SE |
| 0 | 467 | 400 | 26.8 | 3.3 | 31.3 | 3.4 |
| 0.1 | 507 | 446 | 27.0 | 3.1 | 30.7 | 3.2 |
| 1 | 502 | 437 | 33.7² | 3.4 | 38.7² | 3.4 |
| 10 | 444 | 378 | 22.1 | 3.1 | 25.9 | 3.3 |

¹= μg AOS per ml medium
²p < 0.05

A significantly higher proportion of oocytes developed to transferable quality embryos in the 1 μg/ml AOS treatment group. The 0.1 μg/ml AOS had no effect on development, while the higher level (10 μg/ml AOS) had a negative effect. The addition of 1 μg/ml of AOS to the culture medium had a beneficial effect on embryo quality, resulting in a 7% increase in transferable embryos produced.

Although the Invention has been described by way of example, it should be appreciated that variations and modifications may be made without departing from the scope of the Invention as defined in the claims. Furthermore, where known equivalents exist to specific features, such equivalents are incorporated as if specifically referred in this specification.

1. The use of allene oxide synthase as a preservative for semen.

2. The use as claimed in claim 1, wherein the allene oxide synthase is, or has been cloned from, allene oxide synthase from Parthenlum argentatum or is a functionally equivalent variant thereof.

3. The use as claimed in claim 1 or claim 2, wherein the allene oxide synthase has the amino add sequence of SEQ ID No. 1 or is a functionally equivalent variant thereof.

4. The use as claimed in any one of claims 1 to 3, wherein the allene oxide synthase is in a pH-buffered aqueous medium.

5. The use as claimed in claim 4, wherein the allene oxide synthase is present in the medium at a concentration in the range 1 to 10 μg/ml.

6. The use as claimed in any one of claims 1 to 5, wherein the semen is bovine semen.

7. The use as claimed in any one of claims 1 to 5, wherein the semen is human semen.

8. A method of extending the life of semen comprising contacting the semen with allene oxide synthase.

9. A method as claimed in claim 8, wherein the semen is stored in the presence of allene oxide synthase.

10. A method as claimed in claim 8 or claim 9, wherein allene oxide synthase is added to the semen and the semen is then frozen.

11. A method as claimed in claim 10, wherein the frozen semen is thawed before use.

12. A method as claimed in claim 9, wherein the semen is fresh semen.

13. A method as claimed in claim 12, wherein at least 50% of sperm in the semen are viable 4 days from ejaculation.

14. Semen containing allene oxide synthase.

15. The semen as claimed in claim 14, which is frozen semen.

16. The semen as claimed in claim 14, which is fresh semen.

17. The use of the semen of any one of claims 14 to 16 in an artificial insemination process, an in vitro fertlllsatlon process, or a sperm sorting process.

18. The use as claimed in claim 17, where the artificial insemination process includes the step of artificially inseminating a human female, a bovine cow, a horse, a pig, a sheep, or poultry.

19. A composition for preserving semen which comprises allene oxide synthase.


SEQUENCE LISTING (1 sequence)

SEQ ID NO: 1 - protein, 473 aa, Parthenium argentatum
MDPSSKPLREIPGSYGIPFFQPIKDRLEYFYGTGGRDEYFRSRMQKYQSTVFRANMPPGPFVSSNPKVIVLLDAKSFPILFDVSKVEKKDLFTGTYMPSTKLTGGYRVLSYLDPSEPRHAQLKNLLFFMLKNSSNRVIPQFETTYTELFEGLEAELAKNGKAAFNDVGEQAAFRFLGRAYFNSNPEETKLGTSAPTLISSWVLFNLAPTLDLGLPWFLQEPLLHTFRLPAFLIKSTYNKLYDYFQSVATPVMEQAEKLGVPKDEAVHNILFAVCFNTFGGVKILFPNTLKWIGLAGENLHTQLAEEIRGAIKSYGDGNVTLEAIEQMPLTKSVVYESLRIEPPVPPQYGKAKSNFTIESHDATFEVKKGEMLFGYQPFATKDPKVFDRPEEYVPDRFVGDGEALLKYVWWSNGPETESPTVENKQCAGKDFVVLITRLFVIELFRRYDSFEIELGESPLGAAVTLTFLKRASI